(19) United States
(12) Patent Application Publication
(10) Pub. No.: US 2026/0199222 A1
(43) Pub. Date: Jul. 16, 2026

(54) PERSONAL CARE COMPOSITION COMPRISING A VOLATILE ALKANE MIXTURE AND RESINS

(71) Applicant: MOMENTIVE PERFORMANCE MATERIALS INC., Niskayuna, NY (US)

(72) Inventors: Jingya DAI, Shanghai (CN); Amar PAWAR, Tarrytown, NY (US); Benjamin FALK, Tarrytown, NY (US); Roy Rojas WAHL, Tarrytown, NY (US)

(73) Assignee: MOMENTIVE PERFORMANCE MATERIALS INC., Niskayuna, NY (US)

(21) Appl. No.: 19/135,770

(22) PCT Filed: Dec. 5, 2022
(86) PCT No.: PCT/CN2022/136538

§ 371 Date: Jun. 4, 2025

(51) Int. Cl. A61K 8/891; A61Q 1/06; A61Q 1/10

(52) U.S. Cl. CPC A61K 8/891; A61Q 1/06; A61Q 1/10

(57) ABSTRACT

A personal care composition comprises: (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile resins selected from the group consisting of a silicone resin, a hydrocarbon resin, an organic resin, a fluorinated resin, and combinations thereof.

### FIELD OF THE DISCLOSURE

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile resins selected from the group consisting of a silicone resin, a hydrocarbon resin, an organic resin, a fluorinated resin, and combinations thereof. The present disclosure also relates to the linear alkanes described above comprising about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

### BACKGROUND

Resins, alone or combined with plastisizer, are commonly used for skin care, color cosmetics and sun care applications as film formers for water resistance and long wear. However, the processibility of resins in cosmetically acceptable carriers, such as cyclomethicone, is difficult and requires long time to dissolve the resin powder in a silicone-based solvent. However, hydrocarbon based solvents such as isododecane can dissolve the resin powder easily. But the feel of personal care formulations is uncomfortable due to excessive quick drying of isododecane. Therefore, there is a need to develop personal care formulations comprising alkane mixtures which can dissolve resin powder easily and also provide excellent sensory benefits.

### BRIEF SUMMARY OF THE DISCLOSURE

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile resins selected from the group consisting of a silicone resin, a hydrocarbon resin, an organic resin, a fluorinated resin, and combinations thereof.

In some aspects, the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

In some aspects, the resin is a silicone resin comprising monofunctional siloxane units (M or (R¹)₃SiO_{1/2} units), difunctional siloxane units (D or (R²)₂SiO_{2/2} units), trifunctional siloxane units (T or R³SiO_{3/2} units), quattrofunctional siloxane units (Q or SiO_{4/2} units), or combinations thereof, where each of R¹, R², and R³ is independently an alkyl group with 1 to 8 carbon atoms, or an aryl group. In some aspects, the resin is a MQ silicone having a M/Q ratio of about 3 to about 0.3. In some aspects, the resin is a MT silicone having a M/T ratio of about 1 to about 0.01. In some aspects, the resin is a MQ silicone comprising one or more fluorinated moieties. In some aspects, the resin is a MT silicone comprising one or more fluorinated moieties. In some aspects, the resin is an organic resin comprising homopolymers or copolymers of vinyl polymers, acrylic polymers, methacrylic polymers, polyalkylenes, polyvinylpyrrolidones (PVP), or styrenic polymers, or combinations thereof.

In other aspects, the resin is an organic resin comprising polymeric gums, cellulose, cellulose derivatives, agar, pectin, gelatin, starch, starch derivatives, chitosan, chitosan derivatives, or combinations thereof. In some aspects, the resin is a film former.

### BRIEF DESCRIPTION OF THE FIGURES

The accompanying figures, which are incorporated herein, form part of the specification and illustrate embodiments of the present disclosure. Together with the description, the figures further serve to explain the principles of and to enable a person skilled in the relevant art(s) to make and use the disclosed embodiments. The figures are intended to be illustrative, not limiting.

FIG. 1 shows the mixture solution of 1 g of trimethylsiloxysilicate (MQ resin) with 4 g of Alkane Mixture Example 2, and trimethylsiloxysilicate is completely dissolved in an alkane mixture.

FIG. 2 shows the mixture solution of 1 g of trimethylsiloxysilicate (MQ resin) with 4 g of cyclopentasiloxane, and whitish-clumpy residues of trimethylsiloxysilicate are still prominently visible and not readily dissolved in cyclopentasiloxane.

FIG. 3 shows the weight losses of a solution of trimethylsiloxysilicate (MQ resin) in Alkane Mixture Example 2 and a solution of trimethylsiloxysilicate (MQ resin) in isododecane.

FIG. 4(A) shows a film after evaporation of a solution of trimethylsiloxysilicate (MQ resin) in isododecane. FIG. 4(B) shows a film after evaporation of a solution of trimethylsiloxysilicate (MQ resin) in Alkane Mixture Example 2.

### DETAILED DESCRIPTION

As used above, and throughout the description, the following terms, unless otherwise indicated, shall be understood to have the following meanings.

Unless stated otherwise, the terms “a” and “an” and “the” and similar references used in the context of describing a particular aspect of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” encompasses the range of experimental error that occurs in any measurement.

The term, “hydrocarbon” as used herein refers to any chemical structure containing hydrogen atoms and carbon atoms.

The term “alkyl” means any monovalent, saturated straight chain or branched chain hydrocarbon group; and the term “alkane” means organic compounds consisting entirely of single-bonded carbon and hydrogen atoms. Representative examples of alkyls include, but are not limited to, methyl, ethyl, propyl and isobutyl. Examples of alkanes include, but are not limited to, methane, ethane, propane and isobutane.

The term “aryl” includes any aromatic hydrocarbon from which one hydrogen atom has been removed. Specific, non-limiting examples of aryl groups include phenyl and naphthalenyl.

The term “linear C₉-C₁₈ alkanes” means linear alkanes having 9 to 18 alkanes. Examples of linear C₉-C₁₈ alkanes include, but are not limited to, n-decane, n-dodecane, and n-tetradecane.

The term “branched C₁₀-C₁₄ alkane” means branched alkanes having 10 to 14 carbon atoms. Examples of branched C₁₀-C₁₄ alkane include, but are not limited to, isodecane and isododecane.

Other than in the working examples or where otherwise indicated, all numbers expressing amounts of materials, reaction conditions, time durations, quantified properties of materials, and so forth, stated in the specification and claims are to be understood as being modified in all instances by the term “about”.

It will be understood that any numerical range recited herein includes all sub-ranges with that range and any combination of the various endpoints of such ranges or sub-ranges.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

### I. Volatile Alkane Mixtures

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile resins selected from the group consisting of a silicone resin, a hydrocarbon resin, an organic resin, a fluorinated resin, and combinations thereof.

In some aspects, the volatile alkane mixture comprises about 10% to about 90% by weight of linear C₉-C₁₈ alkanes, about 15% to about 90% by weight of linear C₉-C₁₈ alkanes, about 20% to about 90% by weight of linear C₉-C₁₈ alkanes, about 25% to about 85% by weight of linear C₉-C₁₈ alkanes, about 30% to about 85% by weight of linear C₉-C₁₈ alkanes, about 35% to about 80% by weight of linear C₉-C₁₈ alkanes, about 40% to about 80% by weight of linear C₉-C₁₈ alkanes, about 45% to about 75% by weight of linear C₉-C₁₈ alkanes, about 50% to about 75% by weight of linear C₉-C₁₈ alkanes, about 55% to about 70% by weight of linear C₉-C₁₈ alkanes, or about 60% to about 70% by weight of linear C₉-C₁₈ alkanes relative to the total weight of the alkane mixture.

In some aspects, the volatile alkane mixture comprises about 30% to about 85% by weight of linear C₉-C₁₈ alkanes relative to the total weight of the alkane mixture.

In some aspects, the volatile alkane mixture comprises about 50% to about 75% by weight of linear C₉-C₁₈ alkanes relative to the total weight of the alkane mixture.

In some aspects, the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

In some aspects, the linear alkanes comprise about 0% to about 5% by weight of n-decane, about 1% to about 4% by weight of n-decane, or about 2% to about 3% by weight of n-decane relative to the total weight of the alkane mixture. In some aspects, the n-decane is about 0% by weight, about 1% by weight, about 2% by weight, about 3% by weight, about 4% by weight, or about 5% by weight relative to the total weight of the alkane mixture. In some aspects, the n-decane is about 1% by weight relative to the total weight of the alkane mixture. In some aspects, the n-decane is about 5% by weight relative to the total weight of the alkane mixture.

In some aspects, the linear alkanes comprise about 50% to about 80% by weight of n-dodecane, about 55% to about 75% by weight of n-dodecane, about 60% to about 70% by weight of n-dodecane, or about 65% to about 70% by weight of n-dodecane relative to the total weight of the alkane mixture. In some aspects, the n-dodecane is about 50% by weight, about 55% by weight, about 60% by weight, about 65% by weight, about 70% by weight, about 75% by weight, or about 80% by weight relative to the total weight of the alkane mixture. In some aspects, the n-dodecane is about 60% by weight relative to the total weight of the alkane mixture. In some aspects, the n-dodecane is about 65% by weight relative to the total weight of the alkane mixture. In some aspects, the n-dodecane is about 70% by weight relative to the total weight of the alkane mixture.

In some aspects, the linear alkanes comprise about 1% to about 5% by weight of n-tetradecane, about 2% to about 4% by weight of n-tetradecane, or about 3% to about 4% by weight of n-tetradecane relative to the total weight of the alkane mixture. In some aspects, the n-tetradecane is about 1% by weight, about 2% by weight, about 3% by weight, about 4% by weight, or about 5% by weight relative to the total weight of the alkane mixture. In some aspects, the n-tetradecane is about 1% by weight relative to the total weight of the alkane mixture. In some aspects, the n-tetradecane is about 5% by weight relative to the total weight of the alkane mixture.

In some aspects, the linear alkanes comprise about 0% to about 5% by weight of n-hexadecane, about 1% to about 4% by weight of n-hexadecane, or about 2% to about 3% by weight of n-hexadecane relative to the total weight of the alkane mixture. In some aspects, the n-hexadecane is about 0% by weight, about 1% by weight, about 2% by weight, about 3% by weight, about 4% by weight, or about 5% by weight relative to the total weight of the alkane mixture. In some aspects, the n-hexadecane is about 0% by weight relative to the total weight of the alkane mixture. In some aspects, the n-hexadecane is about 1% by weight relative to the total weight of the alkane mixture. In some aspects, the n-hexadecane is about 5% by weight relative to the total weight of the alkane mixture.

In some aspects, the linear alkanes comprise about 0% to about 5% by weight of n-octadecane, about 1% to about 4% by weight of n-octadecane, or about 2% to about 3% by weight of n-octadecane relative to the total weight of the alkane mixture. In some aspects, the n-octadecane is about 0% by weight, about 1% by weight, about 2% by weight, about 3% by weight, about 4% by weight, or about 5% by weight relative to the total weight of the alkane mixture. In some aspects, the n-octadecane is about 0% by weight relative to the total weight of the alkane mixture. In some aspects, the n-octadecane is about 1% by weight relative to the total weight of the alkane mixture. In some aspects, the n-octadecane is about 5% by weight relative to the total weight of the alkane mixture.

In some aspects, the linear alkanes are of plant origin. In some aspects, the linear alkane of plant origin is n-decane. In some aspects, the linear alkane of plant origin is n-dodecane. In some aspects, the linear alkane of plant origin is n-tetradecane. In some aspects, the linear alkane of plant origin is n-hexadecane. In some aspects, the linear alkane of plant origin is n-octadecane.

In some aspects, the linear alkanes are free of alkanes with odd number carbon atoms. In some aspects, the linear alkane is n-decane. In some aspects, the linear alkane is n-dodecane. In some aspects, the linear alkane is n-tetradecane. In some aspects, the linear alkane is n-hexadecane. In some aspects, the linear alkane is n-octadecane.

In some aspects, the volatile alkane mixture comprises about 10% to about 90% by weight of branched C₁₀-C₁₄ alkanes, about 10% to about 85% by weight of branched C₁₀-C₁₄ alkanes, about 10% to about 80% by weight of branched C₁₀-C₁₄ alkanes, about 15% to about 75% by weight of branched C₁₀-C₁₄ alkanes, about 15% to about 70% by weight of branched C₁₀-C₁₄ alkanes, about 20% to about 65% by weight of branched C₁₀-C₁₄ alkanes, about 20% to about 60% by weight of branched C₁₀-C₁₄ alkanes, about 25% to about 55% by weight of branched C₁₀-C₁₄ alkanes, about 25% to about 50% by weight of branched C₁₀-C₁₄ alkanes, about 30% to about 45% by weight of branched C₁₀-C₁₄ alkanes, or about 30% to about 40% by weight of branched C₁₀-C₁₄ alkanes relative to the total weight of the alkane mixture. In some aspects, the volatile alkane mixture comprises about 15% to about 70% by weight of branched C₁₀-C₁₄ alkanes relative to the total weight of the alkane mixture. In some aspects, the volatile alkane mixture comprises about 25% to about 50% by weight of branched C₁₀-C₁₄ alkanes relative to the total weight of the alkane mixture.

### II. Resins

In some aspects, the resin is a silicone resin comprising monofunctional siloxane units (M or (R¹)₃SiO_{1/2} units), difunctional siloxane units (D or (R²)₂SiO_{2/2} units), trifunctional siloxane units (T or R³SiO_{3/2} units), quattrofunctional siloxane units (Q or SiO_{4/2} units), or combinations thereof, where each of R¹, R², and R³ is independently an alkyl group with 1 to 8 carbon atoms, or an aryl group.

In some aspects, the resin is a silicone resin comprising monofunctional siloxane units (M units) having alkyl and/or aryl substituents on silicone atoms, difunctional siloxane units (D units) having alkyl and/or aryl substituents on silicone atoms, trifunctional siloxane units (T units) having alkyl and/or aryl substituents on silicone atoms, quattrofunctional siloxane units (Q units) having alkyl and/or aryl substituents on silicone atoms, or combinations thereof.

In some aspects, the resin is a MQ silicone having a M/Q ratio of about 5 to about 0.1, about 4.5 to about 0.15, about 4 to about 0.2, about 3.5 to about 0.25, about 3 to about 0.4, about 2.5 to about 0.5, about 2 to about 0.6, about 1.2 to about 0.7, or about 1 to about 0.8. In some aspects, the resin is a MQ silicone having a M/Q ratio of about 3 to about 0.3. In some aspects, the resin is a MQ silicone having a M/Q ratio of about 1.2 to about 0.5. Examples of MQ resin include but are not limited to MQ resins with INCI (International Nomenclature Cosmetic Ingredient) names Trimethylsiloxysilicate and are commercially available as SR1000 resin by Momentive Performance Materials, Belsil TMS 803 by Wacker, or DOWSIL™ MQ-1600 Solid Resin by Dow.

In some aspects MQ resin is dissolved in a solvent. In some aspects the solvents are silicone, organic or mixtures thereof. Examples of such resins include but are not limited to SS4230 (Cyclopentasiloxane (and) Trimethylsiloxysilicate), SS4267 (Dimethicone (and) Trimethylsiloxysilicate), Silsoft 74 fluid (Trimethylsiloxysilicate (and) Isododecane) from Momentive Performance Materials.

In some aspects, the resin is a MQ silicone comprising one or more fluorinated moieties. In some aspects MQ resin is dissolved in a solvent. In some aspects the solvents are silicone, organic or mixtures thereof.

In some aspects, the resin is a MT silicone having a M/T ratio of about 2 to about 0.001, about 1.9 to about 0.002, about 1.8 to about 0.003, about 1.7 to about 0.004, about 1.6 to about 0.005, about 1.5 to about 0.006, about 1.4 to about 0.007, about 1.3 to about 0.008, about 1.2 to about 0.009, about 1.1 to about 0.01, about 1 to about 0.01, about 0.9 to about 0.02, about 0.8 to about 0.02, about 0.7 to about 0.03, about 0.6 to about 0.03, about 0.5 to about 0.04, about 0.4 to about 0.05, about 0.3 to about 0.06, about 0.2 to about 0.07, or about 0.1 to about 0.08. In some aspects, the resin is a MT silicone having a M/T ratio of about 1 to about 0.01. In some aspects, the resin is a MT silicone having a M/T ratio of about 0.4 to about 0.05. Examples of MT resin include but are not limited to MT resins with INCI (International Nomenclature Cosmetic Ingredient) names Polymethylsilsesquioxane and are commercially available as SilForm Flexible resin by Momentive Performance Materials.

In some aspects MT resin are dissolved in a solvent. In some aspects solvents are silicone, organic or mixtures thereof.

In some aspects, the resin is a MT silicone comprising one or more fluorinated moieties. Examples of fluoro modified MT resin include but are not limited to MT resins with INCI (International Nomenclature Cosmetic Ingredient) name Trifluoropropyldimethylsiloxy/Trimethylsiloxy Silsesquioxane. In some aspects fluoro-modified MT resin are dissolved in a solvent. In some aspects solvents are silicone, organic or mixtures thereof. Examples of fluoro-modified MT resin include but are not limited to SilForm FR-5 and SilForm FR-10 by Momentive Performance Materials.

In some aspects, the silicone resin comprises about 0% to about 10% by weight of residual silanol, about 0.5% to about 9.5% by weight of residual silanol, about 1% to about 9% by weight of residual silanol, about 1.5% to about 8.5% by weight of residual silanol, about 2% to about 8% by weight of residual silanol, about 2.5% to about 7.5% by weight of residual silanol, about 3% to about 7% by weight of residual silanol, about 3.5% to about 6.5% by weight of residual silanol, about 4% to about 6% by weight of residual silanol, or about 4.5% to about 5.5% by weight of residual silanol relative to the total weight of the silicone resin.

In some aspects, the resin is an organic resin comprising homopolymers or copolymers of vinyl polymers, acrylic polymers, methacrylic polymers, polyalkylenes, polyvinylpyrrolidones (PVP), or styrenic polymers, or combinations thereof.

In some aspects, the resin is an organic resin comprising vinyl homopolymers, vinyl copolymers, acrylic homopolymers, acrylic copolymers, methacrylic homopolymers, methacrylic copolymers, homopolymers of polyalkylenes, copolymers of polyalkylenes, homopolymers of polyvinylpyrrolidones (PVP), copolymers of polyvinylpyrrolidones (PVP), styrenic homopolymers, styrenic copolymers, or combinations thereof.

In some aspects, the copolymers are random copolymers or block copolymers. In some aspects, the copolymers are block copolymers. In some aspects, the block copolymer comprises di-block configuration, tri-block configuration, multi-block configuration, radial-block configuration, star-block configuration, or combinations thereof.

In some aspects, the resin is an organic resin comprising polymeric gums, cellulose, cellulose derivatives, agar, pectin, gelatin, starch, starch derivatives, chitosan, chitosan derivatives, or combinations thereof. In some aspects, the polymeric gums are selected from the group consisting of alginates, carageenans, gum acacia, gum arabic, gum ghatti, gum karaya, gum tragacanth, guar gum, guar hydroxypropyltrimonium chloride, xanthan gum, gellan gum, dammar gum, olibanum gum, and combinations thereof. In some aspects, the cellulose derivatives are selected from the group consisting of sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropylcellulose, methyl cellulose, hydroxypropylmethyl cellulose, microcrystalline cellulose, and combinations thereof. In some aspects, the chitosan derivative is hydroxyethyl chitosan.

In some aspects, the resin is a film former.

In some aspects, the weight ratio of a volatile alkane mixture to non-volatile resins is about 5:95, about 10:90, about 15:85, about 20:80, about 25:75, about 30:70, about 35:65, about 40:60, about 45:55, to about 50:50, about 55:45, about 60:40, about 65:35, about 70:30, about 75:25, about 80:20, about 85:15, about 90:10, or about 95:5.

### III. Personal Care Composition Ingredients

In some aspects, the personal care composition further comprises a silicone wax, an organic wax, or combinations thereof.

In some aspects, the personal care composition further comprises a silicone wax. In some aspects, the silicone wax is selected from the group consisting of C₃₀-C₄₅ alkyl dimethicone, C₃₀-C₄₅ alkyl cetearyl dimethicone crosspolymer, and combinations thereof.

In some aspects, the personal care composition further comprises an organic wax. In some aspects, the organic wax is selected from the group consisting of beeswax, ozokerite wax, carnauba wax, shea butter, stearic acid, cetyl alcohol, and combinations thereof.

In some aspects, the personal care composition further comprises an aqueous phase selected from the group consisting of water, glycerin, butylene glycol, propanediol, xanthan gum, steareth 2, steareth 21, sodium polyacrylate, disodium EDTA, hydroxyethyl urea, a cationic emulsifier, an anionic emulsifier, a non-ionic emulsifier, sodium chloride, and combinations thereof.

In some aspects, the personal care composition further comprises one or more of particulates, pigments, colorants and dyes. In some aspects, the particulates, pigments, colorants and dyes are selected from the group consisting of silica, talc, aluminum starch octenylsuccinate, polymethylsilsesquioxane, nylon-12, starch particles including tapioca starch and rice starch, cellulose particles, cellulose acetate particles, synthetic dyes including azo dyes, lakes, carmines and botanicals, iron oxide, zinc oxide, titanium dioxide, boron nitride, mica, organo-modified hectorite clay, and combinations thereof. In some aspects, the particulates, pigments, colorants and dyes are surface treated with silanes, organofunctional silanes, or combinations thereof.

In some aspects, the personal care composition further comprises one or more preservatives. In some aspects, the preservative is phenoxyethanol, ethylhexylglycerin, or combinations thereof.

In some aspects, the personal care composition further comprises one or more fragrances.

In some aspects, the personal care composition further comprises one or more non-volatile oils selected from the group consisting of a silicone oil, a hydrocarbon oil, an organic oil, a fluorinated oil, and combinations thereof.

In some aspects, the non-volatile oil is silicone with viscosity at 25° C. from about 10 cP to about 200,000,000 cP, about 100 cP to about 150,000,000 cP, about 1,000 cP to about 100,000,000 cP, about 2,000 cP to about 50,000,000 cP, about 3,000 cP to about 10,000,000 cP, about 4,000 cP to about 5,000,000 cP, about 5,000 cP to about 1,000,000 cP, about 6,000 cP to about 500,000 cP, about 7,000 cP to about 100,000 cP, about 8,000 cP to about 50,000 cP, about 9,000 cP to about 40,000 cP, or about 10,000 cP to about 30,000 cP.

In some aspects, the non-volatile oil is a silicone gum selected from the group consisting of polyalkylsiloxane, polyarylsiloxane, polyalkylsilanol, and combinations thereof. In some aspects, the non-volatile oil is selected from the group consisting of an amino modified silicone, a polyether modified silicone, a phenyl modified silicone, and combinations thereof. In some aspects, the non-volatile oil is a phenyl modified silicone selected from the group consisting of phenyltrimethicone, triphenyl dimethylvinyl disiloxane, phenylpropyldimethylsiloxysilicate, and combinations thereof.

In some aspects, the non-volatile oil is an organic oil selected from the group consisting of a C₈-C₄₀ fatty alcohol, an ester of a C₇-C₃₉ fatty acid, an ester of a C₈-C₄₀ fatty alcohol, polyisobutene, a wax, a plant oil including coconut oil, almond oil and avocado oil, a mineral oil, a synthetic oil, and combinations thereof. In some aspects, the organic oil is a C₈-C₄₀ fatty alcohol, a C₁₀-C₄₀ fatty alcohol, a C₁₅-C₃₅ fatty alcohol, a C₂₀-C₃₀ fatty alcohol, or a C₂₅-C₃₀ fatty alcohol. In some aspects, the organic oil is an ester of a C₇-C₃₉ fatty acid, an ester of a C₁₀-C₃₅ fatty acid, an ester of a C₁₅-C₃₀ fatty acid, or an ester of a C₂₀-C₂₅ fatty acid. In some aspects, the organic oil is an ester of a C₈-C₄₀ fatty alcohol, an ester of a C₁₀-C₄₀ fatty alcohol, an ester of a C₁₅-C₃₅ fatty alcohol, an ester of a C₂₀-C₃₅ fatty alcohol, or an ester of a C₂₅-C₃₀ fatty alcohol. In some aspects, the organic oil is polyisobutene. In some aspects, the organic oil is a wax. In some aspects, the organic oil is a plant oil. In some aspects, the plant oil is coconut oil. In some aspects, the plant oil is almond oil. In some aspects, the plant oil is avocado oil. In some aspects, the organic oil is a mineral oil. In some aspects, the organic oil is a synthetic oil. In some aspects, the organic oil is derived from plants.

In some aspects, the non-volatile oil is a sunscreen ingredient selected from the group consisting of avobenzone, octisalate, oxybenzone, homosalate, octocrylene, octyl methoxycinnamate, 2-ethoxyethyl p-methoxycinnamate, ethylhexyl salicylate, ethylhexyl methoxycinnamate, and combinations thereof.

In some aspects, the non-volatile oil is selected from the group consisting of a conditioning agent, an emollient, a sunscreen oil, a natural oil, and combinations thereof.

In some aspects, the personal care composition further comprises one or more humectants selected from the group consisting of propylene glycol, dipropylene glycol, polypropylene glycol, polyethylene glycol, sorbitol, hydroxypropyl sorbitol, hexylene glycol, glycerin, 1,3-butylene glycol, 1,2,6-hexanetriol, ethoxylated glycerin, propoxylated glycerin, and combinations thereof.

In some aspects, the personal care composition is a liquid, an emulsion, a lotion, a cream, an aerosol spray, a solid stick, a gel, a biphasic liquid, or a multi phasic liquid.

In some aspects, the personal care composition is a deodorant, an antiperspirant, a skin cream, a facial cream, a hair shampoo, a hair conditioner, a mousse, a hair styling gel, a hair spray, a protective cream, a lipstick, a lip color, lip gloss, sunscreen, after sun lotion, sun spray, a facial foundations, blushes, makeup, mascara, a skin care lotion, a moisturizer, a facial treatment, eye serum, eye cream, a personal cleanser, a facial cleanser, a bath oil, a perfume, a shaving cream, a pre-shave lotion, an after-shave lotion, a cologne, a sachet, loose facial powder, compact powder, eye shadow, or a sunscreen.

### Example 1

### Preparation of Alkane Mixture Examples 1-3

The mixture of linear alkane of the invention is produced as outlined in EP2417199A1. Linear alkanes and branched C₁₀-C₁₂ alkane were combined and stirred until a homogeneous solution was formed. Table 1 lists the ingredients of alkane mixture examples 1-3.

TABLE 1

| Composition Wt. % | Example 1 | Example 2 | Example 3 |
|---|---|---|---|
| Linear alkane (C₁₀) | 2.8 | 0 | 0.7 |
| Linear alkane (C₁₂) | 64.1 | 69.5 | 61.7 |
| Linear alkane (C₁₄) | 3.4 | 1.3 | 2.1 |
| Linear alkane (C₁₆-C₁₈) | 0 | 2.4 | 4.8 |
| Branched alkane | 29.7 | 26.8 | 30.7 |
| (C₁₀-C₁₂) | | | |

### Example 2

### Preparation of Personal Care Formulation 1

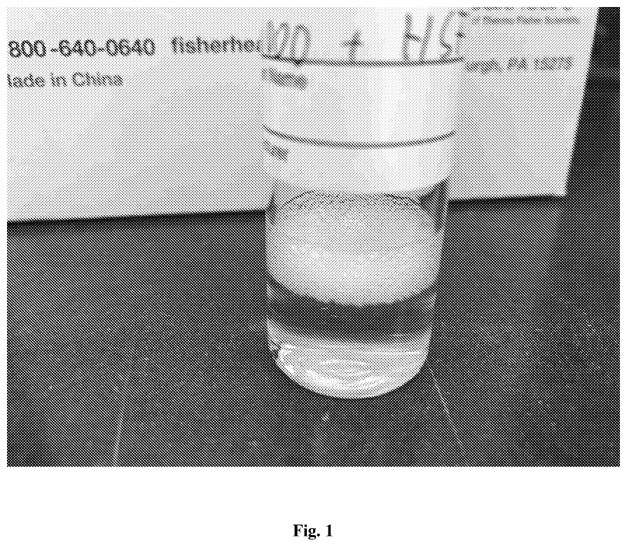

1 g of Trimethylsiloxysilicate (MQ resin) and 4 g of Alkane Mixture Example 2 were mixed using vortex mixer at room temperature and 3000 rpm for 1 min. After standing at room temperature for another minute, the picture of FIG. 1 was taken.

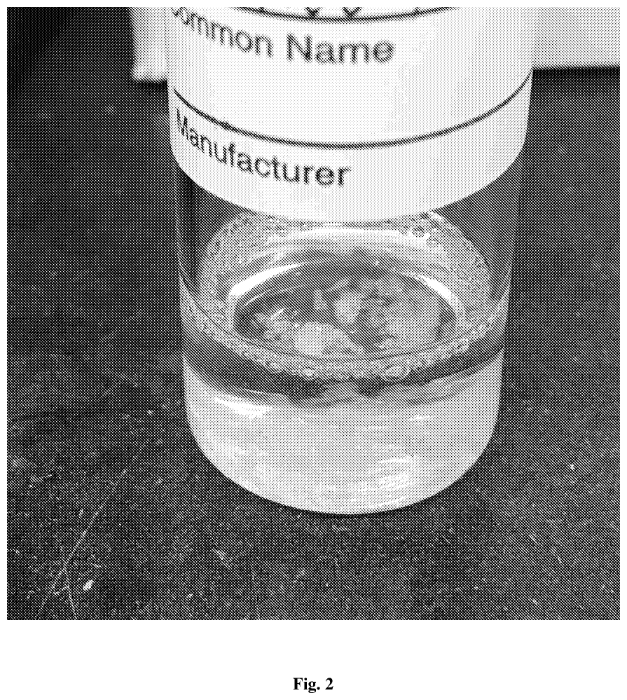

1 g of Trimethylsiloxysilicate (MQ resin) and 4 g of cyclopentasiloxane were mixed using vortex mixer at room temperature and 3000 rpm for 1 min. After standing at room temperature for another minute, the picture of FIG. 2 was taken.

When comparing the pictures in FIG. 1 and FIG. 2, it can clearly be seen that all silicone resin is completely dissolved in an alkane mixture, while whitish-clumpy residues of the silicone resin are still prominently visible and not readily dissolved in cyclopentasiloxane.

The surprising uniqueness of an alkane mixture as a solvent for resins, such as trimethylsiloxysilicate, can be demonstrated when comparing it to a typical alkane solvent such as isododecane, which is often used in the cosmetic industry, especially in make-up.

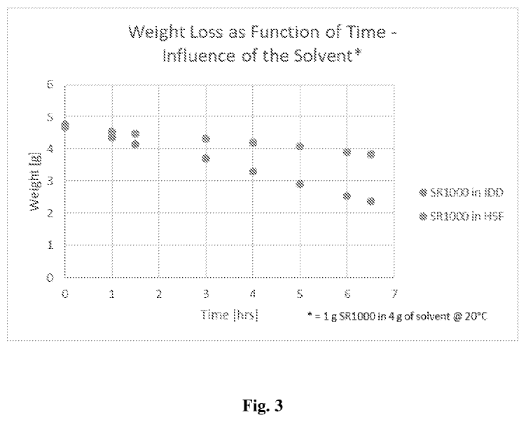

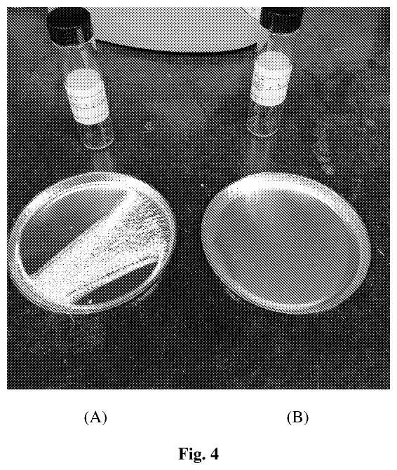

The graph in FIG. 3 demonstrates the weight losses of a solution of trimethylsiloxysilicate (MQ resin) in Alkane Mixture Example 2 with a solution of trimethylsiloxysilicate (MQ resin) in isododecane. The graph in FIG. 3 shows that the isododecane solution has higher weight loss, because isododecane evaporates significantly faster than Alkane Mixture Example 2. This leads to two different films after further evaporation for 24 hrs, as shown in the picture of FIG. 4.

As shown in FIG. 4(A) (trimethylsiloxysilicate (MQ resin) in isododecane), the film is brittle and hard and shows visible cracks. In comparison, as shown in FIG. 4(B) (trimethylsiloxysilicate (MQ resin) in Alkane Mixture Example 2), the film is smooth and soft without any visible cracks. It is widely recognized among experts in the field that a hard and brittle film is disadvantageous in terms of comfort of wear.

These results show that Alkane Mixture Example 2 offers a better benefit to cosmetic formulators as well consumers, such as superior long and comfortable wear.

### Example 3

### Preparation of Personal Care Formulation 2

Table 2 lists the ingredients of Personal Care Formulation 2 (coral shimmer lip gloss). The procedure for preparing Personal Care Formulation 2 is described as follows. Phase A ingredients were combined, heated to 50° C. and stirred until a homogeneous solution was formed. Then, Phase B is added and mixed well.

TABLE 2

| Phase | Ingredient | INCI Name | Wt % |
|---|---|---|---|
| Phase A | Alkane Mixture | Alkane Mixture Example 2 | 20 |
| | Example 2 | | |
| | Petrolatum | Petrolatum | 36 |
| | Microease 1132 | Synthetic Wax (and) | 5 |
| | | Microcrystalline Wax | |
| | Silsoft 034 | Caprylyl Methicone | 20 |
| | Kester Wax K-82-P | C18-38 Alkyl Hydroxystearoyl | 3 |
| | | Stearate | |
| | SR1000 | trimethylsiloxysilicate | 5 |
| Phase B | Coloreze Red Lake | D&C Red 7 Ca Lake (and) Ricinus | 10 |
| | Dispersion LJ | Communis (Castor) Seed Oil | |
| | KTZ Aruban Coral | Mica (and) Titanium Dioxide (and) | 1 |
| | | Iron Oxides [C.I. 77491] | |

### Example 4

### Preparation of Personal Care Formulation 3

Table 3 lists the ingredients of Personal Care Formulation 3 (Long wear lip color). The procedure for preparing Personal Care Formulation 3 is described as follows. Phase A ingredients were combined, heated to 60° C. and stirred until a homogeneous solution was formed. Then, Phase B is added and mixed well.

TABLE 3

| Phase | Ingredient | INCI Name | Wt % |
|---|---|---|---|
| Phase A | BRO-11S2 | Iron Oxide (and) | 6 |
| | | Triethoxycaprylylsilane | |
| | BTD-11S2 | Titanium Dioxide (and) | 4 |
| | | Triethoxycaprylylsilane | |
| | Alkane Mixture Example 2 | Alkane Mixture Example 2 | 20 |
| | Silsoft DML fluid | Dimethicone | 4.4 |
| | Bentone Gel VS-5PC V | Cyclopentasiloxane (and) | 9.3 |
| | | Disteardimonium Hectorite | |
| | | (and) Propylene Carbonate | |
| | Silsoft 1215 fluid | Cyclopentasiloxane (and) | 9.2 |
| | | Dimethiconol | |
| | Velvesil DM gel | Dimethicone (and) Cetearyl | 10 |
| | | Dimethicone Crosspolymer | |
| | Aerosil 200 | Silica | 0.3 |
| | Bentonite | Bentonite | 2 |
| | SilForm Flexible resin | Polymethylsilsesquioxane | 10 |
| | Glycerin | Glycerin | 1 |
| Phase B | SF1642 | C30-45 Alkyl Dimethicone | 11.6 |

### Example 5

### Preparation of Personal Care Formulation 4

Table 4 lists the ingredients of Personal Care Formulation 4 (Long wear Eyeliner). The procedure for preparing Personal Care Formulation 4 is described as follows. Phase A ingredients were combined and stirred until a homogeneous solution was formed. Then, Phase B is added, heated to 50° C. and mixed well. Add Phase C and mix well.

TABLE 4

| Phase | Ingredient | INCI Name | Wt % |
|---|---|---|---|
| Phase A | Dermol 99 | Isononyl Isonononoate | 37 |
| | SR1000 | Trimethylsiloxysilicate | 6 |
| | Silform FR-5 | Trifluoropropyldimethylsiloxy/ | 7 |
| | Fluid | Trimethylsiloxy | |
| | | Silsesquioxane (and) | |
| | | Dimethicone | |
| | Alkane Mixture | Alkane Mixture Example 2 | 5 |
| | Example 2 | | |
| | KTZ Misterioso | Mica (and) Titanium Dioxide | 3 |
| | Pewter | (and) Iron Oxide | |
| | Black Iron | Iron Oxide (and) | 12 |
| | Oxide - | Triethoxycaprylylsilane | |
| | BB0-11S2 | | |
| Phase B | Performalene 500 | Polyethylene | 4.5 |
| | Microease 1132 | Synthetic Wax (and) | 4.5 |
| | | Microcrystalline Wax | |
| | Optiphen | Phenoxyethanol (and) | 1 |
| | | Caprylyl Glycol | |
| Phase C | Silsoft ETS | Ethyl Trisiloxane | 20 |

1. A personal care composition comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile resins selected from the group consisting of a silicone resin, a hydrocarbon resin, an organic resin, a fluorinated resin, and combinations thereof.

2. The personal care composition of claim 1, wherein the volatile alkane mixture comprises about 30% to about 85% by weight of linear alkanes having 9 to 18 carbon atoms relative to the total weight of the alkane mixture.

3. The personal care composition of claim 1, wherein the volatile alkane mixture comprises about 50% to about 75% by weight of linear alkanes having 9 to 18 carbon atoms relative to the total weight of the alkane mixture.

4. The personal care composition of any one of claims 1-3, wherein the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

5. The personal care composition of any one of claims 1-4, wherein the linear alkanes are of plant origin.

6. The personal care composition of any one of claims 1-5, wherein the linear alkanes are free of alkanes with odd number carbon atoms.

7. The personal care composition of any one of claims 1-6, wherein the resin is a silicone resin comprising monofunctional siloxane units (M units), difunctional siloxane units (D units), trifunctional siloxane units (T units), quattrofunctional siloxane units (Q units), or combinations thereof.

8. The personal care composition of any one of claims 1-7, wherein the resin is a silicone resin comprising monofunctional siloxane units (M units) having alkyl and/or aryl substituents on silicone atoms, difunctional siloxane units (D units) having alkyl and/or aryl substituents on silicone atoms, trifunctional siloxane units (T units) having alkyl and/or aryl substituents on silicone atoms, quattrofunctional siloxane units (Q units) having alkyl and/or aryl substituents on silicone atoms, or combinations thereof.

9. The personal care composition of any one of claims 1-8, wherein the resin is a silicone resin comprising monofunctional siloxane units (M or (R¹)₃SiO_{1/2} units), difunctional siloxane units (D or (R²)₂SiO_{2/2} units), trifunctional siloxane units (T or R³SiO_{3/2} units), quattrofunctional siloxane units (Q or SiO_{4/2} units), or combinations thereof, wherein each of R¹, R², and R³ is independently an alkyl group with 1 to 8 carbon atoms, or an aryl group.

10. The personal care composition of any one of claims 1-9, wherein the resin is a MQ silicone having a M/Q ratio of about 3 to about 0.3.

11. The personal care composition of claim 10, wherein the M/Q ratio is about 1.2 to about 0.5.

12. The personal care composition of any one of claims 1-11, wherein the resin is a MQ silicone comprising one or more fluorinated moieties.

13. The personal care composition of any one of claims 1-9, wherein the resin is a MT silicone having a M/T ratio of about 1 to about 0.01.

14. The personal care composition of claim 13, wherein the M/T ratio is about 0.4 to about 0.05.

15. The personal care composition of any one of claims 1-9, 13 and 14, wherein the resin is a MT silicone comprising one or more fluorinated moieties.

16. The personal care composition of any one of claims 1-15, wherein the silicone resin comprises about 0% to about 10% by weight of residual silanol relative to the total weight of the silicone resin.

17. The personal care composition of any one of claims 1-6, wherein the resin is an organic resin comprising homopolymers or copolymers of vinyl polymers, acrylic polymers, methacrylic polymers, polyalkylenes, polyvinylpyrrolidones (PVP), or styrenic polymers, or combinations thereof.

18. The personal care composition of claim 17, wherein the copolymers are random copolymers or block copolymers.

19. The personal care composition of claim 18, wherein the block copolymer comprises di-block configuration, tri-block configuration, multi-block configuration, radial-block configuration, star-block configuration, or combinations thereof.

20. The personal care composition of any one of claims 1-6, wherein the resin is an organic resin comprising polymeric gums, cellulose, cellulose derivatives, agar, pectin, gelatin, starch, starch derivatives, chitosan, chitosan derivatives, or combinations thereof.

21. The personal care composition of claim 20, wherein the polymeric gums are selected from the group consisting of alginates, carageenans, gum acacia, gum arabic, gum ghatti, gum karaya, gum tragacanth, guar gum, guar hydroxypropyltrimonium chloride, xanthan gum, gellan gum, dammar gum, olibanum gum, and combinations thereof; the cellulose derivatives are selected from the group consisting of sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropylcellulose, methyl cellulose, hydroxypropylmethyl cellulose, microcrystalline cellulose, and combinations thereof; and the chitosan derivative is hydroxyethyl chitosan.

22. The personal care composition of any one of claims 1-21, wherein the resin is a film former.

23. The personal care composition of any one of claims 1-22, wherein the weight ratio of a volatile alkane mixture to non-volatile resins is about 10:90 to about 90:10.

24. The personal care composition of any one of claims 1-23, wherein the personal care composition further comprises a silicone wax, an organic wax, or combinations thereof.

25. The personal care composition of claim 24, wherein the silicone wax is selected from the group consisting of C₃₀-C₄₅ alkyl dimethicone, C₃₀-C₄₅ alkyl cetearyl dimethicone crosspolymer, and combinations thereof; and the organic wax is selected from the group consisting of beeswax, ozokerite wax, carnauba wax, shea butter, stearic acid, cetyl alcohol, and combinations thereof.

26. The personal care composition of any one of claims 1-25, wherein the personal care composition further comprises an aqueous phase selected from the group consisting of water, glycerin, butylene glycol, propanediol, xanthan gum, steareth 2, steareth 21, sodium polyacrylate, disodium EDTA, hydroxyethyl urea, a cationic emulsifier, an anionic emulsifier, a non-ionic emulsifier, sodium chloride, and combinations thereof.

27. The personal care composition of any one of claims 1-26, wherein the personal care composition further comprises one or more of particulates, pigments, colorants and dyes.

28. The personal care composition of claim 27, wherein the particulates, pigments, colorants and dyes are selected from the group consisting of silica, talc, aluminum starch octenylsuccinate, polymethylsilsesquioxane, nylon-12, starch particles including tapioca starch and rice starch, cellulose particles, cellulose acetate particles, synthetic dyes including azo dyes, lakes, carmines and botanicals, iron oxide, zinc oxide, titanium dioxide, boron nitride, mica, organo-modified hectorite clay, and combinations thereof.

29. The personal care composition of claim 27 or 28, wherein the particulates, pigments, colorants and dyes are surface treated with silanes, organofunctional silanes, or combinations thereof.

30. The personal care composition of any one of claims 1-29, wherein the personal care composition further comprises one or more non-volatile oils selected from the group consisting of a silicone oil, a hydrocarbon oil, an organic oil, a fluorinated oil, and combinations thereof.

31. The personal care composition of claim 30, wherein the non-volatile oil is silicone with viscosity from about 10 cP to about 200,000,000 cP at 25° C.

32. The personal care composition of claim 30 or 31, wherein the non-volatile oil is a silicone gum selected from the group consisting of polyalkylsiloxane, polyarylsiloxane, polyalkylsilanol, and combinations thereof.

33. The personal care composition of any one of claims 30-32, wherein the non-volatile oil is selected from the group consisting of an amino modified silicone, a polyether modified silicone, a phenyl modified silicone, and combinations thereof.

34. The personal care composition of any one of claims 30-33, wherein the non-volatile oil is a phenyl modified silicone selected from the group consisting of phenyltrimethicone, triphenyl dimethylvinyl disiloxane, phenylpropyldimethylsiloxysilicate, and combinations thereof.

35. The personal care composition of claim 30, wherein the non-volatile oil is an organic oil selected from the group consisting of a C₈-C₄₀ fatty alcohol, an ester of a C₇-C₃₉ fatty acid, an ester of a C₈-C₄₀ fatty alcohol, polyisobutene, a wax, a plant oil including coconut oil, almond oil and avocado oil, a mineral oil, a synthetic oil, and combinations thereof.

36. The personal care composition of claim 30, wherein the non-volatile oil is a sunscreen ingredient selected from the group consisting of avobenzone, octisalate, oxybenzone, homosalate, octocrylene, octyl methoxycinnamate, 2-ethoxyethyl p-methoxycinnamate, ethylhexyl salicylate, ethylhexyl methoxycinnamate, and combinations thereof.

37. The personal care composition of any one of claims 30-36, wherein the non-volatile oil is selected from the group consisting of a conditioning agent, an emollient, a sunscreen oil, a natural oil, and combinations thereof.

38. The personal care composition of any one of claims 1-37, wherein the personal care composition is a liquid, an emulsion, a lotion, a cream, an aerosol spray, a solid stick, a gel, a biphasic liquid, or a multi phasic liquid.

39. The personal care composition of any one of claims 1-38, wherein the personal care composition is a deodorant, an antiperspirant, a skin cream, a facial cream, a hair shampoo, a hair conditioner, a mousse, a hair styling gel, a hair spray, a protective cream, a lipstick, a lip color, lip gloss, sunscreen, after sun lotion, sun spray, a facial foundations, blushes, makeup, mascara, a skin care lotion, a moisturizer, a facial treatment, eye serum, eye cream, a personal cleanser, a facial cleanser, a bath oil, a perfume, a shaving cream, a pre-shave lotion, an after-shave lotion, a cologne, a sachet, loose facial powder, compact powder, eye shadow, or a sunscreen.
